(19) United States
(12) Patent Application Publication
(10) Pub. No.: US 2007/0172910 A1
(43) Pub. Date: Jul. 26, 2007

(54) Food freshness indicator and food storage systems same

(76) Inventor: Ann L. Nolen, Georgetown, TX (US)

Correspondence Address: DAVID ODELL SIMMONS, 7637 PARKVIEW CIRCLE, AUSTIN, TX, 78731, US

(21) Appl. No.: 11/657,679

(22) Filed: Jan. 24, 2007

Related U.S. Application Data
(60) Provisional application No. 60/761,672, filed on Jan. 24, 2006.

(51) Int. Cl. C12Q 1/04; C12M 3/00

(52) U.S. Cl. 435/34; 435/287.1

(57) ABSTRACT

A freshness indicator comprises a support structure that is substantially impermeable to fluids. A test device is engaged with the support structure and includes a pathogen detection composition. The pathogen detection composition provides a prescribed visual indication when in the presence of a prescribed type of pathogens and/or a by-product thereof. A flow limiting structure extends over the test device and has an entire perimeter edge portion thereof attached to the support structure in a manner whereby the flow limiting structure and the support structure jointly form a fluid resistant interface around a space in which the test device resides. The flow limiting structure substantially allows a portion of the prescribed type of pathogen and/or a portion of the by-product thereof to enter the space in which the test device resides and substantially inhibits any portion of the pathogen detection composition from escaping from the space in which the test device resides.

### CROSS REFERENCE TO RELATED APPLICATIONS

This patent application claims priority to co-pending U.S. Provisional Patent Application having Ser. No. 60/761,672 filed Ser. No. 01/24/2006 entitled “Bac-T Baggie”, having a common applicant herewith and being incorporated herein in its entirety by reference.

### FIELD OF THE DISCLOSURE

The disclosures made herein relate generally to methods and devices for detecting bacteriological pathogens and, more particularly, to methods and devices for detecting and/or indicating spoilage of food items.

### BACKGROUND

It is well known that many food items, if not all food items, are subject to spoilage. Consumption of such spoiled food items can cause serious illness. In some instances (e.g., weak immune system, excessive consumption, failure to seek medical assistance, etc), consumption of such spoiled food items can be fatal or cause permanent disability.

Food that is spoiled to an excessive degree is typically fairly easy to identify either by sight, smell and/or taste. The ability to see, smell and/or taste indication(s) that a food item is excessively spoiled actually makes such excessively spoiled food item less susceptible to being unintentionally consumed. To the contrary, a food item that is at the onset of spoilage or early stages of being spoiled is very susceptible to unintentional consumption because it may not be immediately apparent though sight, smell or taste that the food item is at the onset of spoiling or is spoiled. Because of this, a person may actually consume large quantities of such food items, which increases the potential for serious illness, fatality and/or disability.

Contributing to the unintentional consumption of spoiled food items is the wide variety of food storage systems that are commercially-available to consumers. Examples of such commercially-available food storage systems include, but are not limited to, Zip-Loc™ brand plastic bags, Rubbermaid™ brand containers and Tupperware™ brand containers. In most countries, it is common practice for consumers to store remaining portions of food items (e.g., consumer-prepared left-overs, bulk packaged left-overs, etc) in commercially-available food storage systems. The lack of preservatives in certain food items, inadequate refrigeration temperature, exposure to air, storage duration and other such variables make food items stored in such commercially-available food storage systems particularly susceptible to spoilage.

Therefore, a food freshness indicator that can be used in combination with and/or integrally with commercially available food storage systems is advantageous, desirable and useful.

### SUMMARY OF THE DISCLOSURE

Embodiments of the present invention enable spoiled or spoiling at the onset of becoming spoiled to be readily identified. More specifically, at least some embodiments of the present invention provide visual indication that food within a food storage system is either on the onset of spoilage or has become spoiled. In doing so, embodiments of the present invention advantageously limit the potential for unintentional consumption of spoiled food items.

In one embodiment of the present invention, a food freshness indicator comprises a support structure, a test device and a flow limiting structure. The support structure is substantially impermeable to fluids. The test device is engaged with the support structure and includes at least one pathogen detection composition. The at least one pathogen detection composition provides a prescribed visual indication when in the presence of at least one prescribed type of pathogens and/or a by-product thereof. The flow limiting structure extends over the test device and has an entire perimeter edge portion thereof attached to the support structure in a manner whereby the flow limiting structure and the support structure jointly form a fluid resistant interface around a space in which the test device resides. The flow limiting structure substantially allows a portion of the at least one prescribed type of pathogen and/or a portion of the by-product thereof to enter the space in which the test device resides and substantially inhibits any portion of the at least one pathogen detection composition from escaping from the space in which the test device resides.

In another embodiment of the present invention, a food storage system comprises a food storage article having a food- receiving cavity therein and a food freshness indicator attached to the food storage article within the food-receiving cavity. The food freshness indicator includes at least one pathogen detection composition in contact therewith. The at least one pathogen detection composition provides a prescribed visual indication when subjected to contact by said at least one prescribed type of pathogen and/or a by-product thereof. The food freshness indicator includes a one-way semi-permeable membrane extending over the test strip in a manner whereby exposure the test strip is limited to passage of molecules through the one-way semi-permeable membrane. The one-way semi-permeable membrane substantially allows a portion of said at least one prescribed type of pathogen and/or a portion of the by-product thereof to pass therethrough in a direction toward the test strip and substantially inhibits any portion of said at least one pathogen detection composition to pass therethrough in a direction toward food-receiving cavity.

In another embodiment of the present invention, a food storage system comprises a zippered storage bag, a test strip and a one-way semi-permeable membrane. The zippered storage bag has a food-receiving cavity therein. The test strip is engaged with the zippered storage bag within the food-receiving cavity. The test strip includes a plurality of pathogen detection compositions in contact therewith. Each one of the pathogen detection compositions changes from a respective first color to a respective second color when subjected to contact by at least one prescribed type of pathogen and/or a by-product thereof. The one-way semi-permeable membrane extends over the test strip and has an entire perimeter edge portion thereof attached to the zippered storage bag in a manner whereby the one-way semi-permeable membrane and the zippered storage bag jointly form a fluid resistant interface around the test strip. The one-way semi-permeable membrane substantially allows molecules of the at least one prescribed type of pathogen and/or molecules of the by-product thereof to pass therethrough in a direction toward the test strip and substantially inhibits molecules of the pathogen detection compositions to pass therethrough in a direction toward the test strip.

Turning now to specific aspects of the present invention, in at least one embodiment, a liquid transference device is engaged between the test device and the flow limiting structure.

In at least one embodiment of the present invention, the liquid transference device includes a fluid wicking material.

In at least one embodiment of the present invention, at least one of the pathogen detection compositions is provided in the shape of a symbol or character.

In at least one embodiment of the present invention, the support structure includes opposing surfaces, the test device is engaged with a first one of the surfaces and the support structure is made of a polymeric material through which the at least one pathogen detection composition is visible after the at least one pathogen detection composition provides the prescribed visual indication.

In at least one embodiment of the present invention, the flow limiting structure includes a one-way semi-permeable membrane and the one-way semi-permeable membrane is impermeable with respect to a composition molecule greater in size than a water molecule.

These and other objects, embodiments, advantages and/or distinctions of the present invention will become readily apparent upon further review of the following specification, associated drawings and appended claims.

### BRIEF DESCRIPTION OF THE DRAWINGS

FIG. 1 is an illustration showing a bread bag having an embodiment of a food freshness indicator in accordance with the present invention attached thereto.

FIG. 2 is an illustration showing a zippered storage bag having an embodiment of a food freshness indicator in accordance with the present invention attached thereto.

FIG. 3 is an illustration showing a storage container system having an embodiment of a food freshness indicator in accordance with the present invention disposed therein.

FIG. 4 is an illustration showing a storage container system having a cover with an embodiment of a food freshness indicator in accordance with the present invention attached thereto.

FIG. 5 is a cross-sectional view taken along the line 5-5 in FIG. 2.

FIG. 6 is a fragmentary plan view showing the food freshness indicator, wherein a pathogen detection composition of the food freshness indicator visually indicates a spoiled food condition.

### DETAILED DESCRIPTION OF THE DRAWING FIGURES

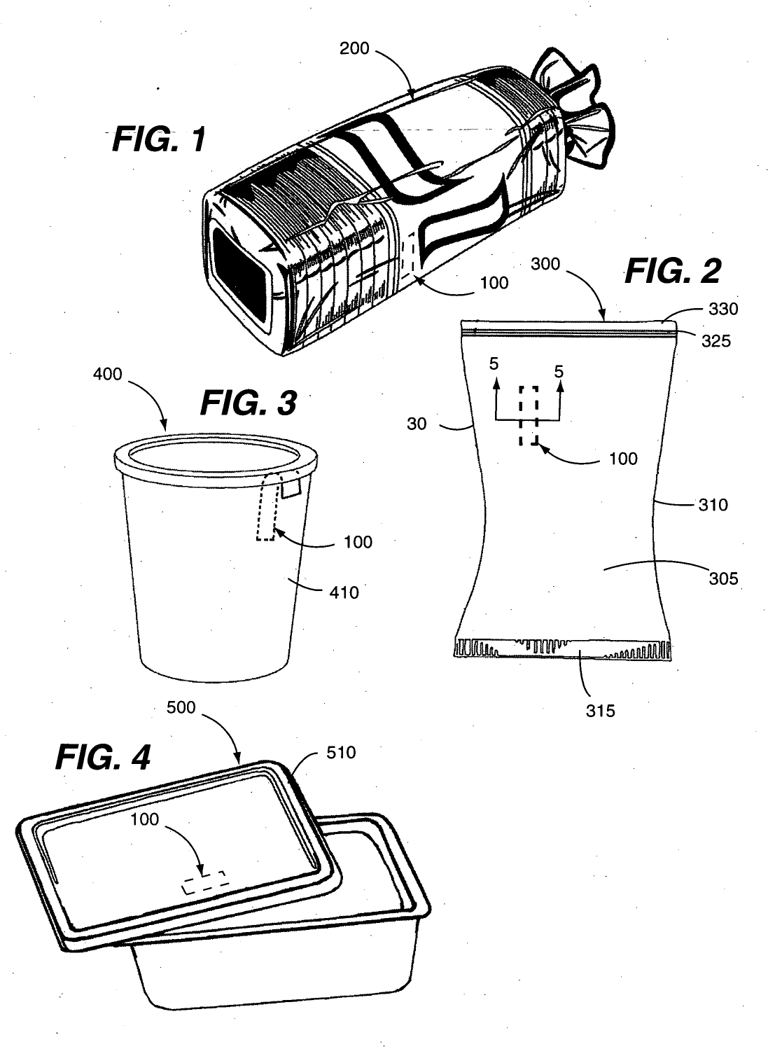

FIGS. 1-4 show various implementation of a food freshness indicator in accordance with the present invention, which is referred to herein as the food freshness indicator 100. FIG. 1 shows a bread bag 200 having the food freshness indicator 100 attached thereto within a food-receiving cavity thereof. FIG. 2 shows a zippered storage bag 300 having the food freshness indicator 100 attached thereto within a food-receiving cavity thereof. FIG. 3 shows a storage container system 400 including a food-receiving body 410 having the food freshness indicator 100 disposed within a food-receiving cavity thereof. FIG. 4 shows a storage container system 500 having a cover 510 with the food freshness indicator 100 attached thereto. The bread bag 200, the zippered storage bag 300 and the storage container articles 400, 500 are examples of food storage systems with which food freshness indicators in accordance with the present invention are useful. It is disclosed herein that a food freshness indicator in accordance with the present invention is not unnecessarily limited to a particular application, use and/or food storage system.

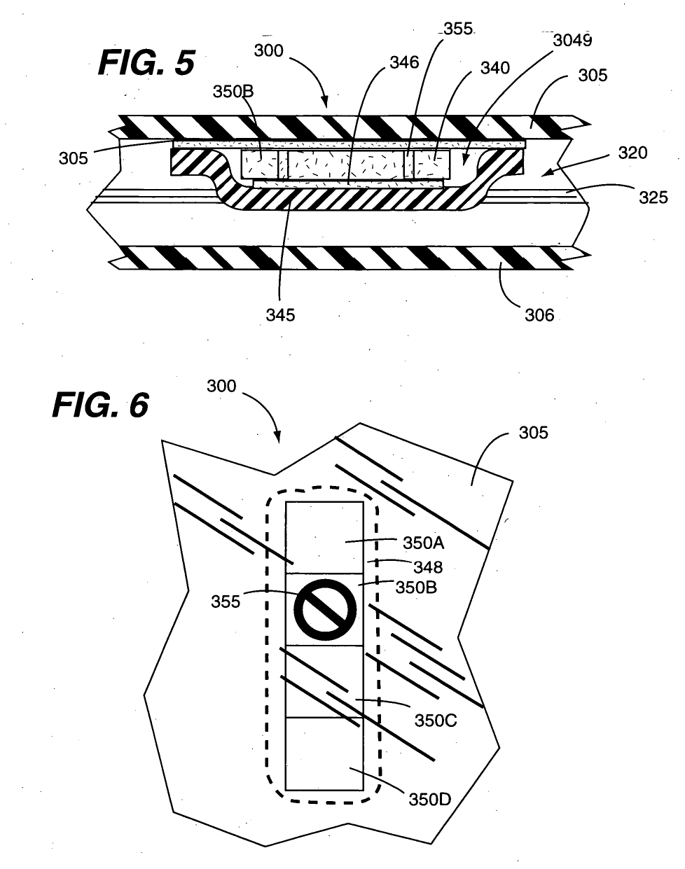

Referring to FIGS. 2 and 5, the zippered storage bag 300 includes walls 305, 306 made of polymeric sheet material. The walls 305, 306 are attached at side interfaces 310 and/or an end interface 315 (depending on the specific manufacturing approach), thereby forming a food-receiving cavity 320. A zipper mechanism 325 is attached between the walls 305, 306 for allowing an open end 330 of the zippered storage bag 300 to be selectively opened and closed. Zip-Loc™ brand plastic bags are one example of commercially-available zippered storage bags.

Referring to FIGS. 5 and 6, the food freshness indicator 100 is attached to zippered storage bag wall 305 within the food-receiving cavity 320. The food freshness indicator 100 includes a support structure 335, a test strip 340, a one-way semi-permeable membrane 345 and a liquid wicking substrate 346. The support structure 335 is substantially impermeable to fluids and is attached to wall 305 by means such as, for example, adhesive, thermal boding, or other suitable method. Polymeric sheet made from materials such as, for example, nylon, polyethylene, polypropylene, and the like are examples of materials from which the support structure may be made. The test device 340 is engaged with the support structure 335. The one-way semi-permeable membrane 345 extends over the test strip 340 and has an entire perimeter edge portion 348 attached to the wall support structure 335 in a manner whereby the one-way semi-permeable membrane 345 and wall 305 jointly form a fluid resistant interface around the test strip 340 such that the flow limiting structure 345 and the support structure 335 jointly form a fluid resistant interface around a space 349 in which the test strip 340 resides. For example, the entire perimeter edge portion 348 can be joined to wall 305 by means such as thermal bonding, laser bonding, adhesive, etc. The fluid wicking substrate 346 resides between the test strip 340 and the one-way semi-permeable membrane 345 for the purpose of encouraging fluid (e.g., fluids that may carry pathogens) to be transported from within the food-receiving cavity 320 to within the space 349 in which the test strip 340 resides. It is disclosed herein that the support structure 335 can be omitted with the one-way semi-permeable membrane 345 extending over the test strip 340 and having the entire perimeter edge portion 348 attached to wall 305 in a manner whereby the one-way semi-permeable membrane 345 and wall 305 jointly form a fluid resistant interface around the test strip 340 such that the flow limiting structure 345 and the support structure 335 jointly form a fluid resistant interface around a space 349 in which the test strip 340 resides.

The test strip 340 includes a plurality of pathogen detection regions 350. Each one of the pathogen detection regions 350 includes a respective pathogen detection composition 355 (shown only for the pathogen detection region 350B). Examples of pathogen detection composition and/or tests corresponding to functionality provided by the respective pathogen detection compositions 355 are presented below. Each one of the pathogen detection compositions detects a respective type or types of pathogen, a respective by-product or by-products thereof or both. As shown with respect to the pathogen detection composition 355 of the pathogen detection region 350B, a prescribed visual indication is evident in response to the pathogen detection composition of the pathogen detection region 350B being subjected to the presence of the respective type or types of pathogen, the respective by-product or by-products thereof or both. The test strip 340 is one embodiment of a test device in accordance with the present invention. It is disclosed herein that, in other embodiments, a test device is in a form other than a strip and may be made from a material other than, for example, a fibrous material (e.g., paper).

In one embodiment, the pathogen detection composition of each pathogen detection region 350 is provided in the shape of a symbol or character. An ‘X’ is an example of such a character and the globally recognized ‘prohibited’ symbol (i.e., a circle with a line through it) is an example of such a symbol. In one embodiment, the pathogen detection composition of some or all of the pathogen detection regions 350 is invisible with respect to a base material from which the test strip 340 is made (e.g., paper or polymer sheet) and becomes visible upon sensed the presence of the respective type or types of pathogen, the respective by-product or by-products thereof or both. Optionally, the pathogen detection composition of some or all of the pathogen detection regions 350 are a first color and turn to a second color upon being subjected to the presence of the respective type or types of pathogen, the respective by-product or by-products thereof or both.

The one-way semi-permeable membrane 345 substantially allows a portion of the prescribed type of pathogen and/or a portion of the by-product thereof to enter the space 349 in which the test strip 340 resides and substantially inhibits any portion of the pathogen detection compositions from escaping from the space 349 in which the test strip resides. More specifically, in one embodiment, the one-way semi-permeable membrane 345 substantially allows molecules of one or more prescribed types of pathogens and/or molecules of one or more type of by-products thereof to pass therethrough in a direction toward the test strip and substantially inhibits molecules of the pathogen detection compositions to pass therethrough in a direction toward the one-way semi-permeable membrane 345. One-way semi-permeable membrane material is commercially available from any number of sources. In one embodiment, the one-way semi-permeable membrane 345 is impermeable with respect to a composition molecule greater in size than a water molecule. The one-way semi-permeable membrane 345 is an embodiment of a flow limiting structure in accordance with the present invention. Optionally, a flow limiting structure in accordance with the present invention may be in the form of a mechanical device (e.g., check valve structure) as opposed to a material that offers one-way semi-permeability on a molecular, macroscopic and/or microscopic level.

The types of pathogens that are desirable to detect are those most responsible for causing gastroenteritis (i.e., food poisoning), which causes diarrhea and in the most extreme of cases can lead to death secondary to dehydration. The specific organisms most responsible for food poisoning include, but are not limited to Salmonella, Shigella, E. Coli, Proteus, Klebsiella, Serrratia, Edwardsiella, Staphylococcus as well as other enteric pathogens such as, for example, Citrobacter and Providence. Because these are living organisms, they cause specific reactions to occur such as the fermentation of lactose, liquefaction of gelatin, Voges-Proskaure, Hydrogen Sulfide, KCN, Phenylalanine deaminase, urease, and gluconate.

Test devices (e.g., test strips) in accordance with the present invention can be configured consistent with conventional test strips based upon, for example, Ames Scientific methods used for detection of micro-organisms in urine. Such conventional test strips are impregnated with an appropriate chemical composition used to effect a color change reaction in response to being exposed to urine containing a respective micro-organism or micro-organisms for which the test detects. If at least one of the particular micro-organisms is present, it causes a color change reaction to occur in a respective chemical composition. Utilizing this same methodology, test strip material impregnated with the appropriate pathogen detection composition will cause a reaction to occur resulting in a visual indication (e.g., a color change), thus indicating that there are pathogens (i.e., bacteriological colonies) present in the food item in sufficient quantities to induce gastroenteritis. In this event, caution should be utilized if the food product is to be consumed.

Presented now are examples of conventional test procedures used for detecting desired pathogens. In one embodiment, a test device in accordance with the present invention is suitably configured to simulate one or more of these conventional test procedures. For example, through encapsulation of reagents with water-soluble coatings, the tests can be implemented in a strip of absorbent material or other form of a test device.

### EXAMPLE 1

### Voges Proskauer Test for Acetylmethyl Carbinol

Reagent: 5% alpha-naphthol in absolute ethyl alcohol and 40% KOH containing 0.3% creatine

Procedure: To 1 ml of a 48 hour old culture grown in MR-VP broth add 0.6 ml 1% alpha-naphthol and 0.2 ml of 40% KOH. Shake well and allow to stand for 5-10 minutes. Development of an orange-red color on the surface of the medium indicates production of acetylmethyl carbinol.

### EXAMPLE 2

### Nitrate Reduction

Reagents: Solution 1—0.8 gm sulfanilic acid in 100 ml of N/5 acetic acid; solution 11—0.5 gm alphanaphthylamine in 100 ml on N/5 acetic acid and solution 111—10% zinc dust in 1% methyl cellulose solution

Procedure: Inoculate nitrate broth and incubate at 370 degrees for up to 5 days in a Durharms fermentation tube. Add 1 ml of a solution 1 followed by solution 11. A red color indicates the presence of nitrites.

### EXAMPLE 3

### Indol

Reagents: Para-dimehtylaminobenzaldehyde 2 gm; Ethyl Alcohol, 95% 190 ml and Hydorchloric Acid, conc. 40 ml

Procedure: Add 1 ml of ether to a 48 hour old culture of organisms in tryptone broth or other appropriate medium and mix. Gently add about 0.5 ml of the reagent down the side of the tube forming a ring between the medium and the ether. Development of a brilliant red ring just below the ether layer indicates production of indol

Kovac's Reagent using amyl or isoamyl alcohol is added to a culture grown in tryptophane broth. Again a deep red color indicates the presence of indol. Tryptophane base and paraa-dimethylamionbenzaldehyde are available in tablet form from Key Sacientific Produts.

### EXAMPLE 4

### Decarboxylase Activity

Arginine Decarboxylase: Suspected organisms are inoculated into Falkow or Moeller arginine broth and incubated 24 hours at 32-35 C. Positive test appears as a purple color indicating decarboxylation of arginine. A negative resul show presence of a yellow color.

Ornithine Decarboxylase. As above except using ornithine broth. Other tests that may be included are well known and have been utilized in the microbacterial laboratory for identification of pathogens. All involve a color change reaction occurring.

### EXAMPLE 5

### Phenylalanine Test

Reagent: Dissolve 3.0 gm ammonium sulfate and 1 ml 10 H2SO4 in 5 ml of 50% saturated ferric ammonium sulfate solution.

Procedure: Inoculate phenylalanine agar with organisms. Add 5 drops of the above reagent. Presence of a green color indicates the presence of Proteus of Providence.

### EXAMPLE 6

### Oxidase Test

A solution of 1% para-aminodimethylaniline monohydrochloride is used. Place a few drops of the solution on suspected colonies and observe for color changes. Oxidase positive colonies become pink changing to red and finally black.

### EXAMPLE 7

### Mannitol

Presence of organism detects the presence of carbohydrates in extract broth with Andrade's indicator.

In general, test for pathogens rely on the presence of moisture for activation. Accordingly, in some embodiments, it is imperative that food items tested using a food freshness indicator in accordance with the present invention be moist and in contact with the flow limiting structure (e.g., the one-way semi-permeable membrane material 345). In order for this to occur, a user will sometimes be required to face the food item so that it contacts the flow limiting structure in such a manner as to allow contact. For instance, once the food item is placed into the zippered storage bag 300, the user presses excess air from the zippered storage bag 300 and gently pats the zippered storage bag 300 onto the food item so that adequate contact is made between the food item and the one-way semi-permeable membrane material 345.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes may be made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A food freshness indicator, comprising:
a support structure that is substantially impermeable to fluids;
a test device engaged with the support structure, wherein the test device includes at least one pathogen detection composition and wherein said at least one pathogen detection composition provides a prescribed visual indication of the presence of at least one prescribed type of pathogen and/or a by-product thereof; and
a flow limiting structure extending over the test device and having an entire perimeter edge portion thereof attached to the support structure in a manner whereby the flow limiting structure and the support structure jointly form a fluid resistant interface around a space in which the test device resides, wherein the flow limiting structure substantially allows a portion of said at least one type of pathogen and/or a portion of the by-product thereof to enter the space in which the test device resides and substantially inhibits any portion of said at least one pathogen detection composition from escaping from the space in which the test device resides.

2. The food freshness indicator device of claim 1, further comprising:
a liquid transference device engaged between the test device and the flow limiting structure.

3. The food freshness indicator device of claim 1 wherein said at least one pathogen detection composition is provided in the shape of a symbol or character.

4. The food freshness indicator device of claim 1 wherein:
the support structure includes opposing surfaces;
the test device is engaged with a first one of said surfaces; and
the support structure is made of a polymeric material through which said at least one pathogen detection composition is visible after said at least one pathogen detection composition provides the prescribed visual indication.

5. The food freshness indicator device of claim 1 wherein:
the flow limiting structure includes a one-way semi-permeable membrane; and
the one-way semi-permeable membrane is impermeable with respect to a composition molecule greater in size than a water molecule.

6. The food freshness indicator device of claim 1, further comprising:
a liquid transference device engaged between the test device and the flow limiting structure;
wherein said at least one pathogen detection composition is provided in the shape of a symbol or character;
the flow limiting structure includes a one-way semi-permeable membrane; and
the one-way semi-permeable membrane is impermeable with respect to a composition molecule greater in size than a water molecule.

7. The food freshness indicator device of claim 6 wherein:
the support structure includes opposing surfaces;
the test device is engaged with a first one of said surfaces; and
the support structure is made of a polymeric material through which said at least one pathogen detection composition is visible after said at least one pathogen detection composition provides the prescribed visual indication.

8. A food storage system, comprising:
a food storage article having a food-receiving cavity therein; and
a food freshness indicator attached to the food storage article within the food-receiving cavity, wherein the food freshness indicator includes at least one pathogen detection composition in contact therewith, wherein said at least one pathogen detection composition provides a prescribed visual indication when subjected to contact by said at least one prescribed type of pathogen and/or a by-product thereof, wherein the food freshness indicator includes a one-way semi-permeable membrane extending over the test strip in a manner whereby exposure the test strip is limited to passage of molecules through the one-way semi-permeable membrane, wherein the one-way semi-permeable membrane substantially allows a portion of said at least one prescribed type of pathogen and/or a portion of the by-product thereof to pass therethrough in a direction toward the test strip and substantially inhibits any portion of said at least one pathogen detection composition to pass therethrough in a direction toward food-receiving cavity.

9. The food storage system of claim 8, further comprising:
a liquid wicking substrate engaged between the test strip and the one-way semi-permeable membrane.

10. The food storage system of claim 8 wherein said at least one pathogen detection composition is provided in the shape of a symbol or character.

11. The food storage system of claim 8 wherein the prescribed visual indication includes a color change of said at least one pathogen detection composition.

12. The food storage system of claim 8 wherein the food storage article is made of a polymeric sheet of material through which said at least one pathogen detection composition is visible after said at least one pathogen detection composition provides the prescribed visual indication.

13. The food storage system of claim 8, further comprising:
a liquid wicking substrate engaged between the test strip and the one-way semi-permeable membrane, wherein the food storage article is made of a polymeric sheet of material through which said at least one pathogen detection composition is visible after said at least one pathogen detection composition provides the prescribed visual indication and wherein the prescribed visual indication includes a color change of said at least one pathogen detection composition

14. A food storage implement, comprising:
a zippered storage bag having a food-receiving cavity therein;
a test strip engaged with the zippered storage bag within the food-receiving cavity, wherein the test strip includes a plurality of pathogen detection compositions in contact therewith and wherein each one of said pathogen detection compositions changes from a respective first color to a respective second color when subjected to contact by at least one prescribed type of pathogen and/or a by-product thereof; and
a one-way semi-permeable membrane extending over the test strip and having an entire perimeter edge portion thereof attached to the zippered storage bag in a manner whereby the one-way semi-permeable membrane and the zippered storage bag jointly form a fluid resistant interface around the test strip, wherein the one-way semi-permeable membrane substantially allows molecules of said at least one prescribed type of pathogen and/or molecules of the by-product thereof to pass therethrough in a direction toward the test strip and substantially inhibits molecules of said pathogen detection compositions to pass therethrough in a direction toward the food-receiving cavity.

15. The food storage system of claim 14 wherein the zippered storage bag is made of a polymeric sheet of material through which each one of the pathogen detection compositions is visible after each one of said pathogen detection compositions changes its respective color.

16. The food storage system of claim 15, further comprising:
a liquid wicking substrate engaged between the test strip and the one-way semi-permeable membrane.

17. The food storage system of claim 16 wherein said at least one pathogen detection composition is provided in the shape of a symbol or character.

18. The food storage system of claim 17 wherein the one-way semi-permeable membrane is impermeable with respect to a composition molecule greater in size than a water molecule.

19. The food storage system of claim 14, further comprising:
a liquid wicking substrate engaged between the test strip and the one-way semi-permeable membrane.

20. The food storage system of claim 19 wherein said at least one pathogen detection composition is provided in the shape of a symbol or character.
